(19) United States
(12) Patent Application Publication
(10) Pub. No.: US 2021/0038690 A1
(43) Pub. Date: Feb. 11, 2021

(54) PROMOTING EPITHELIAL REGENERATION USING HEPARIN BINDING EPIDERMAL GROWTH FACTOR LIKE GROWTH FACTOR

(71) Applicant: The Board of Trustees of the Leland Stanford Junior University, Stanford, CA (US)

(72) Inventors: Peter Luke Santa Maria, Emerald Hills, CA (US); Chloe Domville-Lewis, Nedlands (AU); Daniel Beswick, Menlo Park, CA (US); Robson Capasso, Stanford, CA (US)

(21) Appl. No.: 16/997,607

(22) Filed: Aug. 19, 2020

Related U.S. Application Data
(63) Continuation of application No. 15/492,773, filed on Apr. 20, 2017, now abandoned.
Application No. 15/492,773 is a continuation-in-part of application No. PCT/US2015/056651, filed on Oct. 21, 2015.
(60) Provisional application No. 62/066,564, filed on Oct. 21, 2014.

(51) Int. Cl. A61K 38/18; C07K 14/475; A61K 9/00; A61K 45/06

(52) U.S. Cl. CPC A61K 38/1808; C07K 14/475; A61K 9/0021; A61K 45/06; A61K 9/006; A61K 9/0019

(57) ABSTRACT

Compositions and methods for promoting epithelial tissue regeneration to treat an oral or oropharyygeal wound, e.g., after tonsillectomy are disclosed. In particular, the invention relates to compositions comprising heparin binding epidermal growth factor like growth factor and their use in the treatment of an oral or oropharyngeal wound, e.g., post-therapy treatment to promote epithelial tissue regeneration in wounds, e.g., wounds resulting from surgical removal of tonsil tissue during tonsillectomy procedures, such as palatal, pharyngeal or lingual tonsillectomy. Additionally, HB-EGF can also be used after an adenoidectomy, a procedure sometimes combined with a tonsillectomy, to promote healing of wounds caused by surgical removal of adenoid tissue.

### CROSS-REFERENCE

This application is a Continuation-In-Part of International Application No. PCT/US2015/056651, filed Oct. 21, 2015, which application claims the benefit of U.S. Provisional Application No. 62/066,564, filed Oct. 21, 2014, which applications are incorporated herein by reference in their entirety.

### INCORPORATION BY REFERENCE OF SEQUENCE LISTING PROVIDED AS A TEXT FILE

A Sequence Listing is provided herewith as a text file, “STAN-1409CIP_ST25.txt” created on Apr. 18, 2017 and having a size of 5 KB. The contents of the text file are incorporated by reference herein in their entirety.

### TECHNICAL FIELD

The present invention pertains generally to the treatment of an oral or oropharyngeal wound, e.g., a wound resulting from treatment such as tonsillectomy. In particular, the invention relates to compositions comprising heparin binding epidermal growth factor like growth factor (HB-EGF) and their use in the treatment of an oral or oropharyngeal wound, e.g., the treatment of an oral or oropharyngeal wound post-therapy (e.g., post-operative treatment of tonsillectomy), to promote epithelial tissue regeneration and wound healing.

### BACKGROUND

The following discussion of the background art is intended to facilitate an understanding of the present invention only. The discussion is not an acknowledgement or admission that any of the material referred to is or was part of the common general knowledge as of the priority date of the application.

Over one million tonsillectomies are performed annually in the United States and Europe (Boss et al. (2012) J. Pediatr. 160(5):814-819; Cullen et al. (2009) Natl. Health Stat. Report 2009 (11):1-25; Lafortune et al. (2012) Comparing activities and performance of the hospital sector in Europe: how many surgical procedures performed as in patient and day cases? OECD Health Division Final Report On Work Package II). A significant percentage of patients experience post-tonsillectomy hemorrhage (PTH). PTH can be defined as primary, when it occurs within twenty-four hours of surgery, or secondary, when hemorrhage occurs greater than twenty-four hours after surgery.

Secondary PTH is the most common complication after tonsillectomy and is a significant burden to the health care system. Little progress has been made in decreasing the PTH rate. Most large studies in the U.S. and Europe cite rates of 2-3% for secondary PTH (Collison et al. (2000) 79(8):640-642, 644, 646 passim; Krishna et al. (2001) Laryngoscope 111(8):1358-1361; Lowe et al. (2004) Lancet. 364(9435):697-702; Watson et al. (1993) J. Laryngol. Otol. 107(8):711-715), though some studies report higher rates of PTH (Blakley (2009) Otolaryngol Head Neck Surg. 140(3):288-290; Heidemann et al. (2009) Eur. Arch. Otorhinolaryngol. 266(7):1011-1015; Sarny et al. (2011) Laryngoscope. 121(12):2553-2560). After tonsillectomy, 10% of patients present to the emergency department, and up to 15% are readmitted for observation. Fifty percent of patients with PTH require a procedural intervention to control bleeding, accounting for 3.1% of all tonsillectomies performed. In the United States, one episode of PTH increases the cost of that individual tonsillectomy by more than $2500 (Bhattacharyya et al. (2014) Laryngoscope 124(7):1554-1556; Seshamani et al. (2014) Otolaryngol. Head Neck Surg. 150(4):574-581).

After tonsillectomy, a raw wound is left in the oral cavity to heal by secondary intention. It has been theorized that secondary PTH is due to premature separation of an eschar from the underlying wound, perhaps hastened by underlying infection or dehydration (Johnson et al. (2002) Laryngoscope 112(8 Pt. 2 Suppl. 100):35-36), though this theory is not without controversy. This healing process is poorly understood and there are a lack of animal models in this area to guide future research (Gysin et al. (2013) ORL J. Otorhinolaryngol. Relat. Spec. 75(3):123-132). Currently, there are no treatments available to prevent or decrease the chances of PTH prior to the complication occurring.

Thus, there remains a need for better post-operative care of tonsillectomy to avoid hemorrhage and improve outcome.

### SUMMARY

The present invention is based on the discovery that HB-EGF is useful in the treatment of oral or oropharyngeal wounds, e.g., a wound resulting from therapy, e.g., tonsillectomy, to promote epithelial tissue regeneration and wound healing.

In one aspect, the invention includes a composition comprising HB-EGF for use in treating a surgical wound in a subject caused by a tonsillectomy (e.g., palatal, pharyngeal or lingual tonsillectomy) or adenoidectomy. In one embodiment, the HB-EGF is human HB-EGF. In certain embodiments, the HB-EGF comprises an amino acid sequence selected from the group consisting of SEQ ID NOs:1-4 or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, wherein the HB-EGF variant is capable of binding to and activating an EGF receptor and promoting epitheilial cell proliferation and wound healing. The composition may further comprise a pharmaceutically acceptable excipient. In certain embodiments, the composition further comprises one or more additional agents selected from the group consisting of an antibiotic, an analgesic agent, an anti-inflammatory agent, an anesthetic, and another growth factor. In other embodiments, the composition further comprises one or more substances that decrease neovascularization, increase neoepithelial adherence, or decrease separation of the neoepithelium. In one embodiment, the composition comprises an agent that decreases contraction of underlying muscles (e.g., palatoglossus, palatopharyngeus, tonsillar pillars, base of tongue, or soft palate), whereby separation of the neoepithelium from the underlying musculature decreases.

In another aspect, the invention includes a method of treating a subject after a tonsillectomy, the method comprising administering a therapeutically effective amount of a composition comprising HB-EGF to the subject. The HB-EGF may act to accelerate healing of a surgical wound by stimulating epithelial cell proliferation, increasing the rate of epithelialization, and increasing the thickness of an epithelial layer of a surgical wound.

By “therapeutically effective dose or amount” of a composition comprising HB-EGF is intended an amount that, when administered as described herein, brings about a positive therapeutic response, such as improved wound healing after a tonsillectomy. Improved wound healing after a tonsillectomy may include increasing the speed by which the wound heals, decreasing the amount of new blood vessels that form, which make the wound susceptible to hemorrhage, or reducing the extent of residual scar or keloid or necrotic tissue formation during or after healing of the wound. Additionally, a therapeutically effective dose or amount may reduce or prevent post-tonsillectomy hemorrhage.

The HB-EGF contained in the composition may be pro-HB-EGF or mature HB-EGF. In certain embodiments, the HB-EGF comprises an amino acid sequence selected from the group consisting of SEQ ID NOs:1-4 or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, wherein the HB-EGF variant is capable of binding to and activating an EGF receptor and promoting epitheilial cell proliferation and wound healing.

In certain embodiments, the method further comprises treating the subject with one or more other drugs or agents, such as, but not limited to, an antibiotic, an analgesic agent, an anti-inflammatory agent, an anesthetic, and another growth factor. In other embodiments, the method further comprises treating the subject with one or more substances that decrease neovascularization, increase neoepithelial adherence, or decrease separation of the neoepithelium. In one embodiment, the method further comprises treating the subject with an agent that decreases contraction of underlying muscles (e.g., palatoglossus, palatopharyngeus, tonsillar pillars, base of tongue, or soft palate), whereby separation of the neoepithelium from the underlying musculature decreases.

In certain embodiments, a single dose of HB-EGF or multiple therapeutically effective doses of HB-EGF are administered to the subject. If multiple therapeutically effective doses of HB-EGF are administered to the subject, the composition comprising HB-EGF may be administered daily, for example, once a day, twice a day, or three times a day. Alternatively, the composition comprising HB-EGF may be administered intermittently, for example, once or twice weekly or every other week.

In certain embodiments, an adenoidectomy is performed on the subject in addition to the tonsillectomy, and a surgical wound produced by the adenoidectomy is also treated with a therapeutically effective amount of a composition comprising HB-EGF.

Any appropriate mode of administration may be used for treating a subject for an oral or oropharyngeal wound, e.g., a wound created through therapy (e.g., after surgery, chemotherapy, and/or radiation), e.g., a surgical wound produced by a tonsillectomy or an adenoidectomy. In certain embodiments, the composition is administered orally, parenterally, or topically. In other embodiments, the composition is administered locally to an oral or oropharyngeal wound, e.g., a post-therapy wound, e.g., a surgical wound. For example, the composition may be administered by microneedle injection, spraying the composition on the wound, or as a topical paste. The composition may also be administered orally as a wash, gargle, or rinse. Alternatively, the composition may be administered adjacent to the site of the oral or oropharyngeal wound, e.g., a post-therapy wound, e.g., a surgical wound.

In another aspect, the invention includes a method of stimulating epithelial cell proliferation at an oral or oropharyngeal wound, e.g., a post-therapy wound, e.g., a surgical wound produced by a tonsillectomy, in a subject, the method comprising administering an effective amount of HB-EGF to the subject. In certain embodiments, administering the HB-EGF increases the thickness of an epithelial layer at the oral or oropharyngeal wound, e.g., a post-therapy wound, e.g., a surgical wound, and/or the rate of epithelialization at the wound.

In another aspect, the invention includes a method of stimulating epithelial cell proliferation at an oral or oropharyngeal wound, e.g., a post-therapy wound, e.g., a surgical wound produced by an adenoidectomy in a subject, the method comprising administering an effective amount of HB-EGF to the subject. In certain embodiments, administering the HB-EGF increases the thickness of an epithelial layer at the an oral or oropharyngeal wound, e.g., a post-therapy wound (e.g., a surgical wound), and/or the rate of epithelialization at the wound.

These and other embodiments of the subject invention will readily occur to those of skill in the art in view of the disclosure herein.

### BRIEF DESCRIPTION OF THE FIGURES

FIGS. 1A-1D show representative histology images comparing the control group to the treatment group. FIG. 1A shows control day 4 showing areas of granulation tissue (arrow) and epithelial edge (cross). FIG. 1B shows treatment day 4 showing areas of granulation (arrow) and epithelial edge (cross). The granulation layer and epithelial wound edge were found to be greater in the treatment group. FIG. 1C shows control day 7 showing epithelial separation from the fibrous layer beneath. FIG. 1D shows treatment day 8 also showing epithelial separation. Epithelial separation was found to be less (height and width) in the treatment group with a thicker layer of epithelium overlying. Scale bar=10 μm (Magnification 10×)

FIGS. 2A-2D show representative histology images comparing the control group to the treatment group. FIG. 2A shows control day 9 showing areas of vascularization with an open wound. The epithelial edge (cross) approaches the wound centre with spindle cells in the base of the wound aligned and contracted (star). FIG. 2B shows treatment day 9 showing areas of neovascularization (arrow) with a closed wound. FIG. 2C shows control day 12 showing and area of the wound without epithelial covering and areas of neovascularization (arrow). FIG. 2D shows treatment day 12 showing an epithelialized wound. The absent basement membrane (arrow) indicates the area of the previous open wound. Scale bar=10 μm (Magnification 10×).

FIG. 3A provides a photograph of an SDS-PAGE gel at 0.75 μg/lane. Lane 1 on the left is a marker lane. Lane two is native saliva. Lane three demonstrates the HB-EGF that localizes to approximately 12.5 kb. Lanes 4 through 7, which were run at 0 hours, 0.5 hours, 1 hour and 3 hours, respectively, demonstrate the presence degradation products from HB-EGF starting as early as 0.5 hours and much more strongly at 3 hours. Lane 8 demonstrates an acidic environment (pH=3) where the degradation of HB-EGF is slowed compared to physiologic pH. Lane 9 demonstrates Histatin 5, a protein known to degrade in saliva. Lanes 10-13 demonstrate increasing Histatin 5 degradation as time progresses.

FIG. 3B provides a photograph of a Western blot at 20 ng/lane. Lane 1 on the left demonstrates no HB-EGF was identifiable in normal saliva. Lanes 2-5, respectively, demonstrate decreasing amounts of HB-EGF at times 0 hour, 0.5 hours, 1 hour, and 3 hours. Lane 6 demonstrates that at acidic pH, some degradation of HB-EGF is prevented, likely from inhibition of salivary proteases. Lanes 7 and 8 are positive controls for HBEGF. Lanes 9-13 are negative controls for saliva+Histatin.

### DETAILED DESCRIPTION

The practice of the present invention will employ, unless otherwise indicated, conventional methods of medicine, pharmacology, chemistry, biochemistry, molecular biology and recombinant DNA techniques, within the skill of the art. Such techniques are explained fully in the literature. See, e.g. K. J. Lee Essential Otolaryngology: Head and Neck Surgery, Tenth Edition (McGraw-Hill Education/Medical, 10^{th} edition, 2012); E. N. Myers Operative Otolaryngology: Head and Neck Surgery: Expert Consult (Saunders, 2^{nd} edition, 2008); A. L. Lehninger, Biochemistry (Worth Publishers, Inc., current addition); Sambrook et al., Molecular Cloning: A Laboratory Manual (3^{rd} Edition, 2001); Methods In Enzymology (S. Colowick and N. Kaplan eds., Academic Press, Inc.); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a wound” includes two or more wounds, and the like.

A “wound” is a break or discontinuity in the structure of an organ or tissue, including epithelium, connective tissue, and muscle tissue. Examples of wounds include, but are not limited to, skin wounds, bruises, ulcers, bedsores, grazes, tears, cuts, punctures, psoriasis wounds, tympanic membrane perforations, corneal abrasions and disruptions and burns. A wound may be produced by a surgical procedure (e.g., tonsillectomy or adenoidectomy) and refer to the healing area after partial or total removal of a tonsil or adenoid.

“Topical” application refers to non-systemic local administration of an active ingredient to a surface of a wound.

The terms “heparin binding epidermal growth factor,” “heparin binding epidermal growth factor like growth factor” and “HB-EGF” are used interchangeably and encompass any form of HB-EGF, including the immature proprotein form and various active forms produced by proteolytic processing of the proprotein, including membrane-anchored and soluble forms of HB-EGF, as well as biologically active fragments, variants, analogs, and derivatives thereof that retain HB-EGF biological activity (e.g., bind to and activate an EGF receptor or promote epitheilial cell proliferation and wound healing). The term HB-EGF includes endogenously occurring mammalian heparin binding epidermal growth factor, allelic heparin binding epidermal growth factor, functional conservative derivatives of heparin binding epidermal growth factor, functionally active heparin binding growth factor fragments, and mammalian heparin binding epidermal growth factor homologs such as heparin binding growth factor like growth factor. HB-EGF also includes mutant forms of HB-EGF that show enhanced activity, increased stability, higher yield or better solubility. Optionally, a composition comprising heparin binding epidermal growth factor may contain more than one type, derivative or homolog of HB-EGF.

The HB-EGF for use in the methods of the invention may be native, obtained by recombinant techniques, or produced synthetically, and may be from any source. Representative human HB-EGF sequences are presented in SEQ ID NOs:1-4 for the immature proprotein form of HB-EGF and various active forms of HB-EGF produced by proteolytic processing of the proprotein. Additional representative sequences are listed in the National Center for Biotechnology Information (NCBI) database, including HB-EGF sequences from a number of different species. See, for example, NCBI entries: Accession Nos: L17032, L1703, NP 001936, NM_ 001945, NP_037077, NP_990180, NP_001137562, NP_034545, NP_001104696, NP_001093871, XP_003829241, XP_005425426, NP_001244398, XP_014126447, XP_014131937, XP_013998941, XP_005523504, XP_005617336, XP_005617335, XP_005617334, XP_005617333, XP_848614, XP_013914901, XP_013821061, XP_013809984, XP_005382088, XP_005382087, XP_005503713, XP_005327340, XP_005356014, XP_005238935, XP_013047270, XP_012996694, XP_010869528, XP_005065318, XP_003477196, XP_012956154, XP_004841917, XP_004744871, XP_012875794, XP_004696718, XP_004652486, XP_002937773, XP_004610052, XP_004586534, XP_004586533, XP_012697566, XP_003782186, XP_012604548, XP_004686855, XP_012501863, XP_012501862, XP_012501861, XP_004397849, XP_002190931, XP_004280331, XP_003756676, XP_004643289, XP_004477893, XP_003266511, XP_012327017, XP_012006016, XP_012006015, XP_012006014, XP_012006013, XP_004008912, XP_011714646, NP_001158639, and NP_001273220; all of which sequences (as entered by the date of filing of this application) are herein incorporated by reference. Any of these sequences, or a biologically active fragment thereof, or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used to produce a composition comprising HB-EGF as described herein. Additionally, the HB-EGF may comprise post-translational modifications, such as glycosylation or phosphorylation. Although any source of HB-EGF can be utilized to practice the invention, preferably the HB-EGF is derived from a human source, particularly when the subject undergoing therapy is human.

“Function-conservative variants” are proteins in which a given amino acid residue has been changed without altering overall conformation and function of the protein, including, but not limited to, replacement of an amino acid with one having similar properties (such as, for example, acidic, basic, hydrophobic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine.

“Substances that decrease neovascularization” may include biologic and/or non-biologic substances, including, but not limited to, anti-angiogenic factors, such as substances that inhibit vascular endothelial growth factors (VEGF), e.g., bevicuzimab, a recombinant humanized antibody; ranibizumab, a fragment of bevicuzimab; pegaptanib, an aptamer; aflibercept, a recombinant fusion protein comprising binding domains of VEGF receptors; genistein, a flavonoid that possesses anti-angiogenic activity, lornafarnib, an inhibitor of farnesyl transferase, and any other substance that decreases neovascularization.

“Substances that improve adherence or decrease separation of the neoepithelium” include biologic and/or non-biologic substances, including, but not limited to glues or adhesives applied by any manner (e.g., topically or by injection) and heat treated calcium chloride. The term also encompasses substances that maintain or limit degradation of hemidesmosomes or their sub-components, which help mediate adhesion of epithelium to the underlying basement membrane. “Substances that improve adherence or decrease separation of the neoepithelium” may also include biologic or non-biologic materials that decrease muscular contraction of the underlying muscles (e.g., palatoglossus and palatopharyngeus, the tonsillar pillars, or base of tongue or soft palate) to mitigate ‘squeezing off’ of the neoepithelium including, but not limited to, nitric oxide, calcium blocking agents and troponin C inhibitors, which inhibit cross-bridging and force generation.

The term “subject” includes both vertebrates and invertebrates, including, without limitation, mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like.

“Treatment” of a subject or “treating” a subject for a disease or condition herein means reducing or alleviating clinical symptoms of the disease or condition such as impaired or slow wound-healing.

“Promote,” “enhance,” or “improve” wound healing, e.g., wound healing after tonsillectomy, generally means increasing the speed by which the wound heals or reducing the extent of residual scar or keloid or necrotic tissue during or after healing of the wound.

An “effective amount” or a “therapeutically effective amount” means an amount of HB-EGF, or a substance that decreases neovascularization, or a substance that increases neoepithelial adherence, or a substance that decreases contraction of muscles (such as the palatoglossus and palatopharyngeus, the tonsillar pillars, or base of tongue or soft palate), which decreases separation of the neoepithelium from the underlying musculature, wherein the amount is sufficient to enhance epithelial cell proliferation, epithelial regeneration, or wound healing. For example, an effective amount of an active agent can be an amount that results in a local (e.g., in a perforation, wound, or scar area) or systemic level of HB-EGF that exceeds 200 microgram/mi. Alternatively, an effective amount of an agent is an amount that results in a faster healing of a perforation or wound or reduced scar or necrotic tissue formation than in the absence of the agent. An effective amount may also refer to an amount or dose of an active agent or drug sufficient to increase the local and/or systemic levels of HB-EGF by at least 10 to 200 percent, at least 50 to 100 percent, or at least 60 to 80 percent of the level of HB-EGF before administration of the active agent or drug, or any percent within these ranges, such as 10, 15, 20, 25, 30, 35, 40, 45, 50, 55, 60, 65, 70, 75, 80, 85, 90, 95, 100, 125, 150, 175, or 200%.

A therapeutically effective amount can ameliorate or present a clinically significant response in a subject, in that, e.g., post-tonsillectomy wound healing is promoted, or scar formation is reduced. Alternatively, a therapeutically effective amount is sufficient to improve a clinically significant wound healing or scar formation condition in the host.

A “structure,” when referring to delivery of HB-EGF or an inhibitor of HB-EGF, or to delivery of a substance that decreases neoangiogenesis or increases neoepithelial adherence includes, but is not limited to, any scaffold, polymer, construction, fabrication, mounting, support, disc, block, coating, layer, abutment, backing, device, or foam. The term also includes the patient's own tissue, debris, or a graft, which may be used in delivery. In certain embodiments, the structure is applied in a fully formed state or in a state that undergoes a phase change or other change that modifies the structure. For example, a viscous liquid, which is used as structure for delivery, may remain in liquid form or form a solid state after application to a wound.

A “vehicle,” when referring to delivery of HB-EGF or inhibitors of HB-EGF, or to delivery of other aforementioned substances to decrease neoangiogenesis or increase neoepithelial adherence includes, but is not limited to, any polymer, agent, carrier, instrument, operation, medium, apparatus, appliance, contraption, gadget, tool, widget, implement or utensil. The term “vehicle” also refers to any soluble carrier or excipient including, but not limited to saline, buffered saline, dextrose, water, glycerol and combinations thereof. The formulation should suit the mode of administration. Examples of suitable formulations, known in the art, can be found in Remington's Pharmaceutical Sciences (latest edition), Mack Publishing Company, and Easton, Pa.

“Epithelium” refers to the covering of internal and external surfaces of the body, including the lining of vessels and other small cavities. It consists of cells joined by small amounts of cementing substances. Epithelium is classified into types on the basis of the number of layers deep and the shape of the superficial cells. In this context, it refers to the superficial layer of cells covering a wound area, e.g., post-tonsillectomy or post-adenoidectomy wound area.

As used herein, “about” or “approximately” mean within 50 percent, preferably within 20 percent, more preferably within 5 percent, of a given value or range.

A value which is “substantially different” from another value can mean that there is a statistically significant difference between the two values. Any suitable statistical method known in the art can be used to evaluate whether differences are significant or not.

“Statistically significant” difference means a significance is determined at a confidence interval of at least 90%, more preferably at a 95% confidence interval.

The terms “peptide,” “oligopeptide,” and “polypeptide” refer to any compound comprising naturally occurring or synthetic amino acid polymers or amino acid-like molecules including but not limited to compounds comprising amino and/or imino molecules. No particular size is implied by use of the terms “peptide,” “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Preferably, the polypeptide is between about 40 and 300 residues in length. Generally, one skilled in art can easily select the maximum length in view of the teachings herein. Further, peptides and polypeptides, as described herein, for example synthetic peptides, may include additional molecules such as labels or other chemical moieties. Such moieties may further enhance interaction of HB-EGF with an EGF receptor and/or stimulation of epitheilial cell proliferation and/or wound healing, and/or enhance HB-EGF stability or delivery.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, glycosylation, acetylation, phosphorylation, and the like.

Amino acid derivatives can also include modifications to the native sequence, such as deletions, additions and substitutions (generally conservative in nature), so long as the polypeptide or peptide maintains the desired activity (e.g., promote epitheilial cell proliferation and wound healing). These modifications may be deliberate, as through site-directed mutagenesis, or may be accidental, such as through mutations of hosts that produce the proteins or errors due to PCR amplification. Furthermore, modifications may be made that have one or more of the following effects: increasing affinity and/or specificity for an EGF receptor, enhancing epitheilial cell proliferation and/or wound healing, and facilitating cell processing.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full length molecule, but may include at least about 15-25 contiguous amino acid residues of the full length molecule, and can include at least about 20-50, 60-90, or more contiguous amino acid residues of the full length molecule, or any integer between 5 amino acids and the full length sequence, provided that the fragment in question retains biological activity, such as HB-EGF activity, as defined herein (e.g., the ability to bind to and activate an EGF receptor and promote epitheilial cell proliferation and/or wound healing).

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule, which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as the ability to bind to and activate an EGF receptor and promote epitheilial cell proliferation and/or wound healing. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that HB-EGF can be used to promote tissue regeneration and wound healing in oral epithelium (see Example 1). HB-EGF can be used in post-operative treatment to promote epithelial tissue regeneration in wounds resulting from surgical removal of tonsil tissue during tonsillectomy procedures, such as palatal, pharyngeal or lingual tonsillectomy. Additionally, HB-EGF can also be used after an adenoidectomy, a procedure sometimes combined with a tonsillectomy, to promote healing of wounds caused by surgical removal of adenoid tissue.

HB-EGF can be used in methods of treating a subject with an oral or oropharyngeal wound. Such wounds include, but are not limited to, wounds characteristic of or resulting from a treatment or therapy that effects the oral cavity or oropharnynx. Examples of such wound are oral mucositis, e.g., ulceration of mucosal tissue; apthous ulcer; a post-surgical wound in the oral cavity; a gingival wound; a wound in an area surrounding an extracted tooth; and the like. A wound suitable for treatment using HB-EGF can be caused by, e.g., trauma, medical treatment (including surgery, radiation and/or chemotherapy), systemic disease, substance use, an iatrogenic cause, and/or an infection. Accordingly, HB-EGF can be used in the treatment of an oral or oropharyngeal wound post-therapy (e.g. post-surgery, post-radiation therapy, and/or post-chemotherapy). In some embodiments, a subject suitable for treatment with a method of the present disclosure, includes a subject having an an oral or oropharyngeal wound resulting from medical treatment (including surgery, radiation and/or chemotherapy).

In order to further an understanding of the invention, a more detailed discussion is provided below regarding the use of HB-EGF to promote healing after tonsillectomy or adenoidectomy.

A. HB-EGF

As explained above, the methods of the present invention include methods of treating a subject with an oral or oropharyngeal wound by administering HB-EGF to the subject, e.g., post-operative administration of HB-EGF after a tonsillectomy or adenoidectomy. Any form of HB-EGF may be used in the practice of the invention, including the immature proprotein form of HB-EGF and various active forms of HB-EGF produced by proteolytic processing of the proprotein, including membrane-anchored and soluble forms of HB-EGF, as well as biologically active fragments, variants, analogs, and derivatives thereof that retain HB-EGF biological activity (e.g., promote epitheilial cell proliferation and wound healing).

The HB-EGF for use in the methods of the invention may be native, obtained by recombinant techniques, or produced synthetically, and may be from any source. Representative human HB-EGF sequences are presented in SEQ ID NOs:1-4 for the immature proprotein form of HB-EGF and various active forms of HB-EGF produced by proteolytic processing of the proprotein. Additional representative sequences are listed in the National Center for Biotechnology Information (NCBI) database, including HB-EGF sequences from a number of different species. See, for example, NCBI entries: Accession Nos: L17032, L1703, NP_001936, NM_001945, NP_037077, NP_990180, NP_001137562, NP_034545, NP_001104696, NP_001093871, XP_003829241, XP_005425426, NP_001244398, XP_014126447, XP_014131937, XP_013998941, XP_005523504, XP_005617336, XP_005617335, XP_005617334, XP_005617333, XP_848614, XP_013914901, XP_013821061, XP_013809984, XP_005382088, XP_005382087, XP_005503713, XP_005327340, XP_005356014, XP_005238935, XP_013047270, XP_012996694, XP_010869528, XP_005065318, XP_003477196, XP_012956154, XP_004841917, XP_004744871, XP_012875794, XP_004696718, XP_004652486, XP_002937773, XP_004610052, XP_004586534, XP_004586533, XP_012697566, XP_003782186, XP_012604548, XP_004686855, XP_012501863, XP_012501862, XP_012501861, XP_004397849, XP_002190931, XP_004280331, XP_003756676, XP_004643289, XP_004477893, XP_003266511, XP_012327017, XP_012006016, XP_012006015, XP_012006014, XP_012006013, XP_004008912, XP_011714646, NP_001158639, NP_001273220; all of which sequences (as entered by the date of filing of this application) are herein incorporated by reference. Any of these sequences, or a biologically active fragment thereof, or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used to produce a composition comprising HB-EGF as described herein. Additionally, the HB-EGF may comprise post-translational modifications, such as glycosylation or phosphorylation. Although any source of HB-EGF can be utilized to practice the invention, preferably the HB-EGF is derived from a human source, particularly when the subject undergoing therapy is human.

According to various embodiments of the invention, the complete HB-EGF proprotein (SEQ ID NO:1) or any biologically active polypeptide obtained by cleavage of the 208 amino acid proprotein, may be used in the methods described herein. Biologically active fragments of HB-EGF will generally include at least about 40-200 contiguous amino acid residues of the full length HB-EGF proprotein, but may include at least about 60-100 contiguous amino acid residues of the full length molecule, and may include at least about 70-90 or more contiguous amino acid residues of the full length molecule, or any integer between 40 amino acids and the full length sequence, provided that the fragment in question retains biological activity, such as the ability to bind to and activate the EGF receptor. Additionally, HB-EGF polypeptides may stimulate epithelial cell proliferation and healing at an oral or oropharyngeal wound, e.g., a post-therapy wound, e.g., a surgical wound produced by a tonsillectomy or adenoidectomy. In certain embodiments, a polypeptide selected from the group consisting of SEQ ID NOs:2-4 is used in treatment of a wound, e.g., post-operative treatment of a wound.

The compositions useful in the methods of the invention may comprise biologically active variants of HB-EGF, including variants of HB-EGF from any species. Such variants should retain the desired biological activity of the native polypeptide such that the pharmaceutical composition comprising the variant polypeptide has the same therapeutic effect as the pharmaceutical composition comprising the native HB-EGF when administered to a subject. That is, the variant polypeptide will serve as a therapeutically active component in the pharmaceutical composition in a manner similar to that observed for the native HB-EGF. Methods are available in the art for determining whether a variant polypeptide retains the desired biological activity, and hence serves as a therapeutically active component in the pharmaceutical composition. Biological activity can be measured using assays specifically designed for measuring activity of the native HB-EGF, including assays described herein for evaluating the effect of the variant polypeptide on wound healing (see Example 1). Additionally, antibodies raised against a biologically active native HB-EGF polypeptide can be tested for their ability to bind to a variant polypeptide, where effective binding is indicative of a polypeptide having a conformation similar to that of the native HB-EGF.

Suitable biologically active variants of native or naturally occurring HB-EGF can be biologically active fragments, analogs, muteins, and derivatives of the HB-EGF polypeptide, as defined above. For example, amino acid sequence variants of HB-EGF can be prepared by introducing mutations in the cloned DNA sequence encoding the native peptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382;); Sambrook et al. (2001) Molecular Cloning: A Laboratory Manual (Cold Spring Harbor Laboratory Press, 3^{rd} Edition); U.S. Pat. No. 4,873,192; and the references cited therein; herein incorporated by reference. Guidance as to appropriate amino acid substitutions that do not destroy biological activity of a peptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly⇔Ala, Val⇔Ile⇔Leu, Asp⇔Glu, Lys⇔Arg, Asn⇔Gln, and Phe⇔Trp⇔Tyr.

Guidance as to the regions of HB-EGF that can be altered by residue substitutions, deletions, or insertions can be found in the art. See, for example, the structure/function relationships and/or binding studies discussed in Thompson et al. (1994) J. Biol. Chem. 269(4):2541-2549, Nishi et al. (2004) Growth Factors 22(4):253-60, Hung et al. (2014) Biochemistry 53(12):1935-1946, Nanba et al. (2004) Biochem. Biophys. Res. Commun. 320(2):376-82, Higashiyama et al. (1992) J. Biol. Chem. 267 (9):6205-6212, Mitamura et al. (1995) J. Biol. Chem. 270(3):1015-1019, Louie et al. (1998) Mol. Cell 1(1):67-78, Nakamura et al. (2000) J. Biol. Chem. 275(24):18284-18290, Hoskins et al. (2008) Biochem. Biophys. Res. Commun. 375(4):506-511, Zhou et al. (2007) Cell Prolif. 40(2):213-230, Davis-Fleische et al. (2001) Growth Factors 19(2):127-143, and Shin et al. (2003) J. Pept. Sci. 9(4):244-250; the contents of which are herein incorporated by reference in their entireties.

In constructing variants of HB-EGF, modifications are made such that variants continue to possess the desired activity. Obviously, any mutations made in the DNA encoding the variant polypeptide must not place the sequence out of reading frame and preferably will not create complementary regions that could produce secondary mRNA structure.

Biologically active variants of HB-EGF will generally have at least about 70%, preferably at least about 80%, more preferably at least about 90% to 95% or more, and most preferably at least about 98% to 99% or more amino acid sequence identity to the amino acid sequence of a reference HB-EGF peptide molecule (e.g., pro-HB-EGF (SEQ ID NO:1) or a mature form of HB-EGF (SEQ ID NOs:2-4) produced by proteolytic processing of the proprotein), which serves as the basis for comparison. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have the same number of amino acids, additional amino acid residues, or deleted amino acid residues with respect to the reference amino acid sequence. The contiguous segment used for comparison to the reference amino acid sequence will typically include at least 8 contiguous amino acid residues, and may be 10, 12, 13, 17, 36, 40, 50, 60, 70, or more amino acid residues. Corrections for sequence identity associated with conservative residue substitutions or gaps can be made (see, e.g., Smith-Waterman homology search algorithm). A biologically active variant of a native HB-EGF polypeptide of interest may differ from the native polypeptide by as few as 1-20 amino acids, including as few as 1-15, as few as 1-10, such as 6-10, or as few as 5, including as few as 4, 3, 2, or even 1 amino acid residue.

The precise chemical structure of a polypeptide having HB-EGF activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having HB-EGF activity as used herein. Further, the primary amino acid sequence of the polypeptide may be augmented by derivatization using sugar moieties (glycosylation), polyethylene glycol (PEG), or by other supplementary molecules such as lipids, phosphate, acetyl, methyl, or pyroglutamyl groups, and the like. It may also be augmented by conjugation with saccharides. Certain aspects of such augmentation are accomplished through post-translational processing systems of the producing host; other such modifications may be introduced in vitro. In any event, such modifications are included in the definition of an HB-EGF polypeptide used herein as long as the HB-EGF activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of HB-EGF polypeptides of interest as used herein.

The art provides substantial guidance regarding the preparation and use of HB-EGF variants. In preparing HB-EGF variants, one of skill in the art can readily determine which modifications to the native HB-EGF nucleotide or amino acid sequence will result in a variant that is suitable for use as a therapeutically active component of a pharmaceutical composition used in the methods of the present invention. In addition, recombinant HB-EGF is also commercially available, for example, from R&D Systems, Inc. (Minneapolis, Minn.), Sigma-Aldrich (St. Louis, Mo.), and ProSpec (Ness-Ziona, Israel).

B. Production of HB-EGF

HB-EGF can be prepared in any suitable manner (e.g., recombinant expression, purification from cell culture, chemical synthesis, etc.) and in various forms (e.g. native, mutated, glycosylated, phosphorylated, lipidated, fusions, labeled, etc.). HB-EGF polypeptides include naturally-occurring polypeptides, recombinantly produced polypeptides, synthetically produced polypeptides, or polypeptides produced by a combination of these methods. Means for preparing polypeptides are well understood in the art. Polypeptides are preferably prepared in substantially pure form (i.e. substantially free from other host cell or non-host cell proteins).

In one embodiment, the polypeptides are generated using recombinant techniques. One of skill in the art can readily determine nucleotide sequences that encode the desired polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of re-combinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), p1.161 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the HB-EGF polypeptides described herein. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal polypeptide or leader sequence. With the present invention, both the naturally occurring signal polypeptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal polypeptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the polypeptides can be further purified using conventional techniques well known in the art, such as by any of the techniques described above.

HB-EGF polypeptides can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethyl styrene copolymers, divinylbenzene-chloromethyl styrene copolymers and divinylbenzene-benzhydrylaminopolystyrene copolymers.

HB-EGF polypeptides can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

C. Pharmaceutical Compositions

HB-EGF can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the HB-EGF or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include:

polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of HB-EGF (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, topical, or localized delivery.

The pharmaceutical preparations herein can also be housed in a syringe, an implantation device, a microneedle injection system, or the like, depending upon the intended mode of delivery and use. Preferably, the compositions comprising HB-EGF, prepared as described herein, are in unit dosage form, meaning an amount of a conjugate or composition of the invention appropriate for a single dose, in a premeasured or pre-packaged form.

The compositions herein may optionally include one or more additional agents, such as other drugs for treating an oral or oropharyngeal wound, e.g., a wound produced by a tonsillectomy or an adenoidectomy, or other medications used to treat a subject for a condition or disease. Particularly preferred are compounded preparations including HB-EGF and one or more other drugs for treating a post-operative wound, such as, but not limited to, analgesic agents, anesthetic agents, antibiotics, anti-inflammatory agents, substances that decrease neovascularization, substances that increase neoepithelial adherence, substances that decrease separation of the neoepithelium, or other growth factors, or other agents that promote wound healing. Alternatively, such agents can be contained in a separate composition from the composition comprising HB-EGF and co-administered concurrently, before, or after the composition comprising HB-EGF.

D. Administration

At least one therapeutically effective cycle of treatment with HB-EGF will be administered to a subject for treatment of a wound. By “therapeutically effective cycle of treatment” is intended a cycle of treatment that when administered, brings about a positive therapeutic response with respect to treatment of an individual for an oral or oropharyngeal wound, e.g., a post-therapy wound, e.g., a surgical wound produced by a tonsillectomy or an adenoidectomy. Of particular interest is a cycle of treatment with HB-EGF that improves post-operative wound healing. Improved wound healing of an oral or oropharyngeal wound, e.g., after a tonsillectomy or an adenoidectomy may include increasing the speed by which the wound heals, decreasing the amount of new blood vessels that form, which make the wound susceptible to hemorrhage, or reducing the extent of residual scar or keloid or necrotic tissue formation during or after healing of the wound. Additionally, a therapeutically effective dose or amount may reduce or prevent post-operative hemorrhaging.

In certain embodiments, multiple therapeutically effective doses of compositions comprising HB-EGF and/or one or more other therapeutic agents, such as other growth factors or drugs or agents for treating a surgical wound, or other medications will be administered. The compositions of the present invention are typically, although not necessarily, administered orally, via injection (subcutaneously, intravenously, or intramuscularly), by infusion, topically, or locally. Additional modes of administration are also contemplated, such as intra-arterial, pulmonary, transdermal, intradermal, transmucosal, rectal, intravaginal, and so forth.

The preparations according to the invention are also suitable for local treatment. In a particular embodiment, a composition of the invention is used for localized delivery of HB-EGF, for example, for treatment of an oral or oropharyngeal wound, e.g., a post-therapy wound, e.g., a wound produced by a tonsillectomy or an adenoidectomy. Compositions may be administered directly on the surface of a wound or adjacent to a wound. For example, the composition may be administered by microneedle injection, spraying the composition on the wound, or as a topical paste. The composition may also be added to wound dressings. Alternatively, the composition may be administered orally as a wash, gargle, or rinse. The particular preparation and appropriate method of administration are chosen to target the HB-EGF to the site in need of wound healing.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising HB-EGF and other agents may be administered using the same or different routes of administration in accordance with any medically acceptable method known in the art.

In another embodiment, the pharmaceutical compositions comprising HB-EGF and/or other agents are administered prophylactically, e.g., to prevent post-tonsillectomy hemorrhage. Such prophylactic uses will be of particular value for subjects who suffer from a condition which impairs or slows down the healing of an oral or oropharyngeal wound, e.g., a post-therapy wound, e.g., a wound produced by a tonsillectomy or an adenoidectomy.

In another embodiment of the invention, the pharmaceutical compositions comprising HB-EGF and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising HB-EGF as provided herein to a patient suffering from a condition that is responsive to treatment with HB-EGF contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with HB-EGF. More specifically, the compositions described herein are effective in treating an oral or oropharyngeal wound, e.g., a post-therapy wound, e.g., a wound produced by a tonsillectomy or an adenoidectomy.

Those of ordinary skill in the art will appreciate which conditions HB-EGF can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. The amount of HB-EGF administered will depend on the potency of the particular form of HB-EGF (e.g., mature HB-EGF or pro-HB-EGF) and the magnitude of its effect on wound epithelialization and healing and the route of administration.

HB-EGF, prepared as described herein (again, preferably provided as part of a pharmaceutical preparation), can be administered alone or in combination with one or more other therapeutic agents for treating a post-operative wound, such as, but not limited to, analgesic agents, anesthetic agents, antibiotics, anti-inflammatory agents, substances that decrease neovascularization, substances that increase neoepithelial adherence, substances that decrease separation of the neoepithelium, or other growth factors, or other agents that promote wound healing, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

HB-EGF can be administered prior to, concurrent with, or subsequent to other agents. If provided at the same time as other agents, HB-EGF can be provided in the same or in a different composition. Thus, HB-EGF and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising HB-EGF and a dose of a pharmaceutical composition comprising at least one other agent, such as another growth factor or drug for treating a wound, which in combination comprise a therapeutically effective dose, according to a particular dosing regimen. Similarly, HB-EGF and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

E. Kits

The invention also provides kits comprising one or more containers holding compositions comprising HB-EGF, and optionally one or more other drugs for treating an oral or oropharyngeal wound, e.g., a post-therapy wound, e.g., a wound produced by a tonsillectomy or an adenoidectomy, such as, but not limited to, analgesic agents, anesthetic agents, antibiotics, anti-inflammatory agents, substances that decrease neovascularization, substances that increase neoepithelial adherence, substances that decrease separation of the neoepithelium, or other growth factors, or other agents that promote wound healing. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions describing methods for post-therapy care, e.g., post-operative care, of an oral or oropharyngeal wound, e.g., a tonsillectomy wound as described herein. The package insert can be an unapproved draft package insert or can be a package insert approved by the Food and Drug Administration (FDA) or other regulatory body.

### III. EXEMPLARY NON-LIMITING ASPECTS OF THE DISCLOSURE

Certain non-limiting aspects of the disclosure numbered 1-47 are provided below:
1. A method of treating a subject after a tonsillectomy, the method comprising administering a therapeutically effective amount of a composition comprising heparin binding epidermal growth factor (HB-EGF) to the subject.
2. The method of 1, wherein the subject is human.
3. The method of 1, wherein the HB-EGF is human HB-EGF.
4. The method of 1, wherein the HB-EGF promotes epithelialization of a surgical wound produced by the tonsillectomy by stimulating epithelial cell proliferation.
5. The method of 1, wherein treating the subject accelerates healing of a surgical wound produced by the tonsillectomy.
6. The method of 1, wherein treating the subject increases thickness of an epithelial layer at a surgical wound produced by the tonsillectomy.
7. The method of 1, wherein treating the subject increases rate of epithelialization at a surgical wound produced by the tonsillectomy.
8. The method of 1, wherein the composition is administered locally to a surgical wound produced by the tonsillectomy.
9. The method of 8, wherein the composition is administered by microneedle injection.
10. The method of 8, wherein the composition is administered by spraying the composition on the wound.
11. The method of 1, wherein the composition is administered orally, parenterally, or topically.
12. The method of 1, wherein the composition is administered adjacent to the site of a surgical wound produced by the tonsillectomy.
13. The method of 1, further comprising treating the subject with an antibiotic, an analgesic agent, an anti-inflammatory agent, an anesthetic, or another growth factor.
14. The method of 1, further comprising treating the subject with a substance that decreases neovascularization or a substance that improves adherence or decreases separation of the neoepithelium.
15. The method of 14, wherein the substance decreases contraction of palatoglossus or palatopharyngeus muscles, whereby separation of the neoepithelium from the underlying musculature decreases.
16. The method of 1, wherein the composition further comprises a pharmaceutically acceptable carrier.
17. The method of 16, wherein the carrier is selected from the group consisting of an aqueous solution, a gel, a lotion, a balm, or a paste.
18. The method of 1, wherein multiple therapeutically effective doses of the HB-EGF are administered to the subject.
19. The method of 18, wherein multiple cycles of treatment are administered to the subject for a time period sufficient to effect at least a partial healing of the wound.
20. The method of 19, wherein the time period is at least 2 to 5 days.
21. The method of 20, wherein the time period is at least a week.
22. The method of 21, wherein the time period is at least 2 weeks.
23. The method of 19, wherein multiple cycles of treatment are administered to the subject for a time period sufficient to effect a complete healing of the wound.
24. The method of 1, wherein the composition comprises a sustained-release formulation or is administered using a sustained-release device.
25. The method of 1, wherein a single dose of HB-EGF is administered to the subject.
26. The method of 1, wherein the tonsillectomy is a palatal, pharyngeal, or lingual tonsillectomy.
27. The method of 1, further comprising performing an adenoidectomy.
28. The method of 27, further comprising treating a surgical wound produced by the adenoidectomy with a therapeutically effective amount of the composition comprising HB-EGF.
29. The method of 28, wherein the composition is administered by microneedle injection into the surgical wound produced by the adenoidectomy.
30. The method of 28, wherein the composition is administered by spraying the composition on the wound produced by the adenoidectomy.
31. The method of 28, wherein the composition is administered adjacent to the site of the surgical wound produced by the adenoidectomy.
32. The method of 28, wherein the HB-EGF comprises an amino acid sequence having at least 70% identity to an amino acid sequence selected from the group consisting of SEQ ID NOs:1-4, wherein the HB-EGF is capable of stimulating epithelial cell proliferation at a surgical wound produced by an adenoidectomy.
33. The method of 32, wherein the HB-EGF comprises an amino acid sequence having at least 80% identity to an amino acid sequence selected from the group consisting of SEQ ID NOs:1-4, wherein the HB-EGF is capable of stimulating epithelial cell proliferation at a surgical wound produced by an adenoidectomy.
34. The method of 33, wherein the HB-EGF comprises an amino acid sequence having at least 90% identity to an amino acid sequence selected from the group consisting of SEQ ID NOs:1-4, wherein the HB-EGF is capable of stimulating epithelial cell proliferation at a surgical wound produced by an adenoidectomy.
35. The method of 34, wherein the HB-EGF comprises an amino acid sequence selected from the group consisting of SEQ ID NOs:1-4.
36. A method of stimulating epithelial cell proliferation at a surgical wound produced by a tonsillectomy in a subject, the method comprising administering an effective amount of HB-EGF to the subject.
37. The method of 36, wherein administering the HB-EGF increases thickness of an epithelial layer at the surgical wound.
38. The method of 36, wherein administering the HB-EGF increases rate of epithelialization at the wound.
39. A method of stimulating epithelial cell proliferation at a surgical wound produced by an adenoidectomy in a subject, the method comprising administering an effective amount of HB-EGF to the subject.
40. The method of 39, wherein administering the HB-EGF increases thickness of an epithelial layer at the surgical wound.
41. The method of 39, wherein administering the HB-EGF increases rate of epithelialization at the wound.
42. The method of 39, wherein the composition is administered locally to a surgical wound produced by the adenoidectomy.
43. The method of 42, wherein the composition is administered by microneedle injection.
44. The method of 42, wherein the composition is administered by spraying the composition on the wound.
45. The method of 39, wherein the composition is administered orally, parenterally, or topically.
46. The method of 39, wherein the composition is administered adjacent to the site of a surgical wound produced by the adenoidectomy.
47. A composition comprising heparin binding epidermal growth factor (HB-EGF) for use in treating a surgical wound in a subject caused by a tonsillectomy or adenoidectomy.

### IV. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Post Tonsillectomy Secondary Hemorrhage: Epithelial Separation in a Mouse Tongue Model and Potential for Prevention Using Heparin Binding Epidermal Growth Factor Like Growth Factor

Post-tonsillectomy wound healing and factors that contribute to post-tonsillectomy hemorrhage (PTH) have not been fully elucidated. This study sought to assess oral wound healing in a mouse model and investigate whether a growth factor (GF) may be a potential preventative measure for PTH.

Methods

All animal work was approved by Stanford University's Administrative Panel on Laboratory Animal Care. All mice used for all experiments were 6-10 weeks old female CBA/CAJ (15-25 g) mice purchased from Jackson Laboratories (Florida, USA). All surgical interventions were performed using inhaled isoflurane at 3-4% for induction and 1-2% for maintenance.

Creation of Tongue Wound

After administering inhaled anesthesia, a standardized wound using a 2 mm punch biopsy (Miltex, Plainsboro, N.J.) to create the wound made in the anterior tongue, adjacent to the tip on the lateral margin, of each mouse down to the level of the tongue musculature under microscopic visualization.

Treatment Groups

Mice were randomized in an unblended fashion into two groups, a treatment group and a control group. Each group comprised 42 mice, allowing for the sacrifice of three mice daily from Day 1 to Day 14.

Treatment group mice were administered HB-EGF (5 μg/ml, Prospec Bio) daily via intramuscular injection into the base of the wound. Control group mice were administered sterile saline daily via intramuscular injection into the base of the wound. Injections in both groups were performed daily starting on Day 0, the day of the procedure, to Day 14, the conclusion of the data gathering. In all mice, HB-EGF or saline soaked gelfoam was placed into the wound immediately after injecting the base as the mouse recovered from anesthesia.

Animal Sacrifice and Harvesting of Tissue

The sacrifice and tissue harvest schedule was the same in the treatment and control groups. In both groups, three mice were sacrificed each day starting on Day 1, the first day following the procedure, to Day 14. Sacrifice was performed via cervical dislocation under anesthesia. Immediately following sacrifice, tongue containing the healing wound was excised and fixed in formalin for histology.

Histology

Histology was performed on the tongue samples according to a previously published technique (Santa Maria et al. (2015) Tissue Eng. Part A. 21(9-10):1483-1494; Santa Maria et al. (2010) Laryngoscope 120(10):2061-2070; herein incorporated by reference). Briefly, the entire wound area was harvested using a 4 mm punch biopsy with the wound area centred in the tissue sample. Sections were cut perpendicular to the plane of the tongue wound.

Wound Assessment

Microscopic photographs of each wound were prepared. The photographs were coded, and the inventors evaluating the photographs were blinded to if the histological images were from treatment or control groups, as well as to each other's response. Each wound was assessed based on the following criteria:

wound bed open or closed,

epithelial thickness,

keratin thickness,

granulation tissue thickness,

presence of neovascularization,

separation of epithelium from underlying basement membrane, and

spindle cell proliferation and contraction.

The photographs were then uncoded, and the results were combined.

Statistical Analysis

Statistical analysis was performed using STATA 13.1 software. Differences in thicknesses were analysed using a two tailed paired t-test for comparison of means, and Pearson's Chi-square testing was used to compare epithelial separation, wound closure and wound reopening on given days post biopsy.

Results

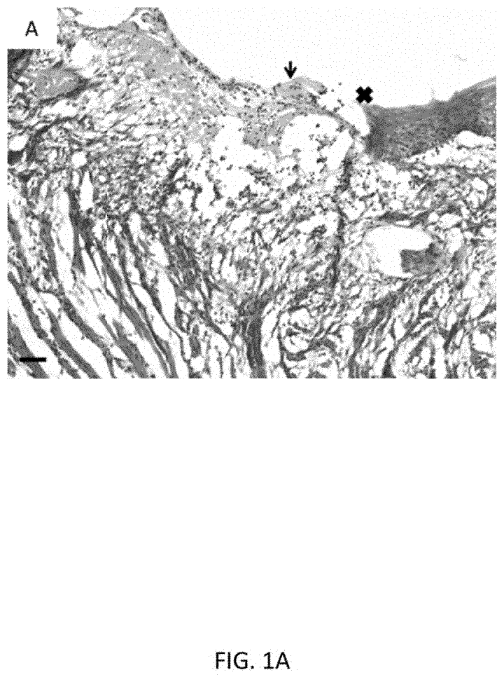

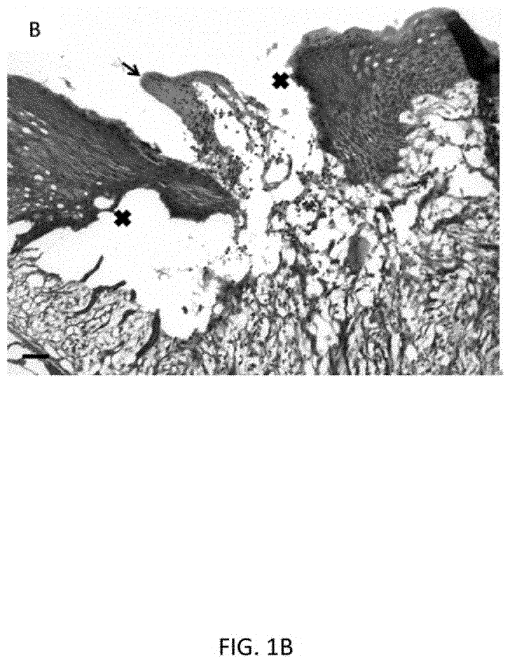

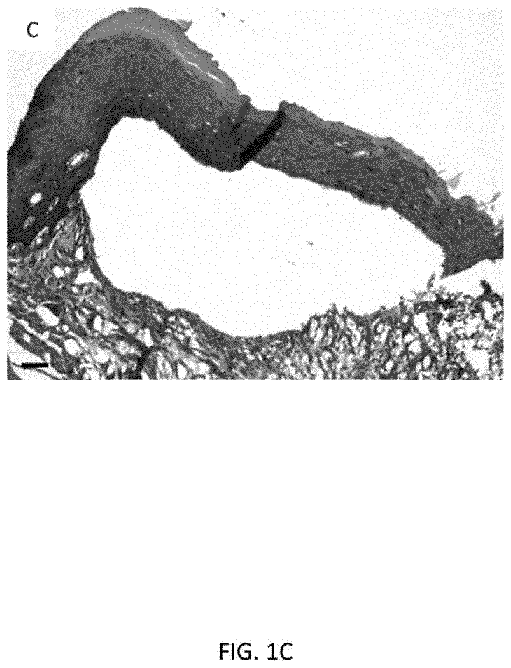

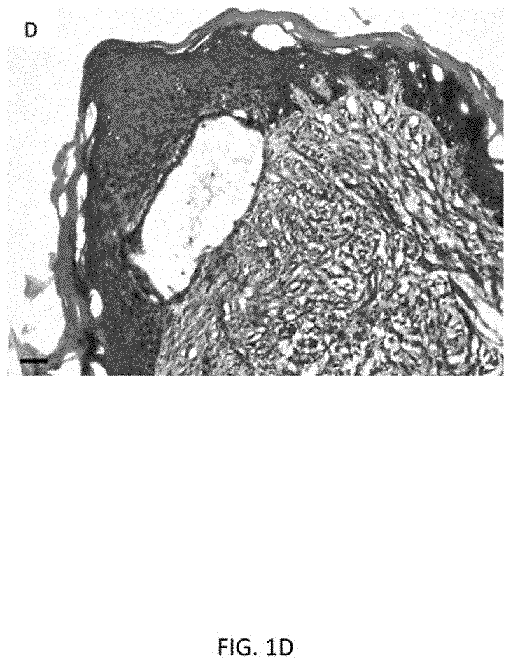

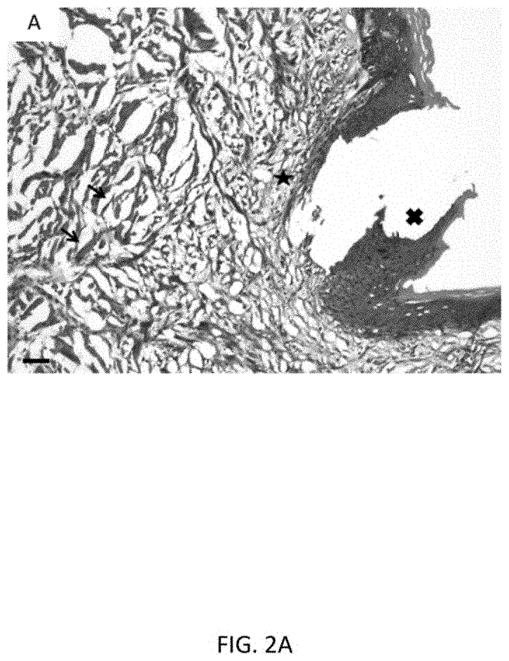

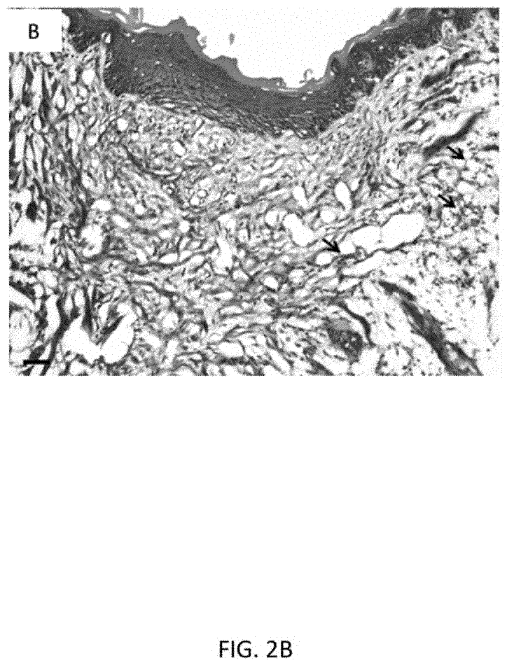

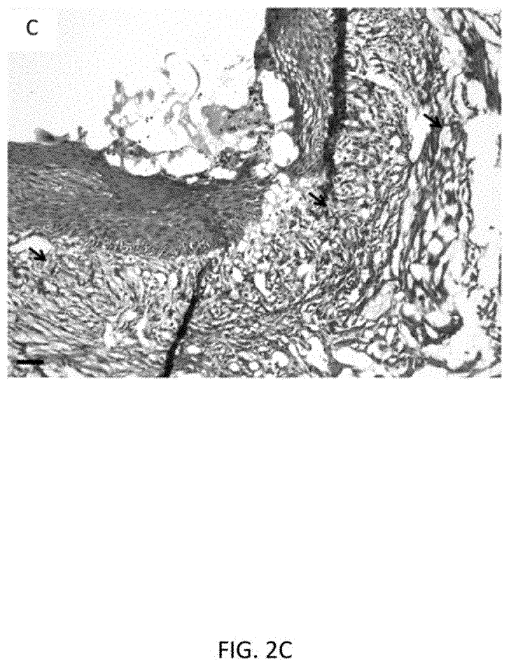

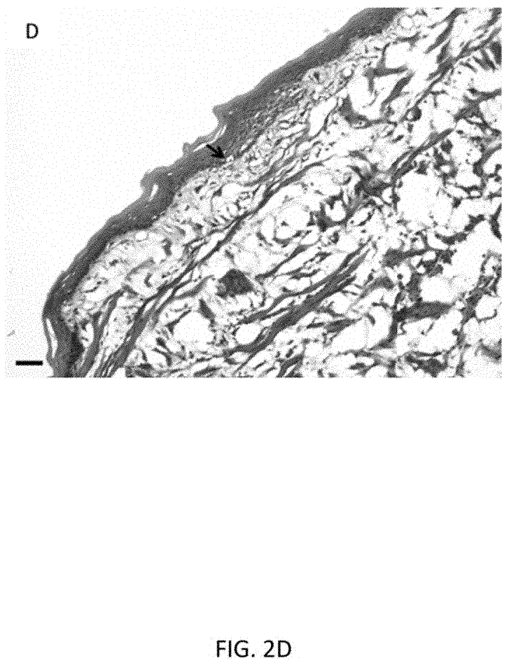

The protocol was carried out without deviation. Results are shown in FIGS. 1A-1D and FIGS. 2A-2D and summarized in Table 1. Compared to the control group, wounds in the experimental group injected with HB-EGF showed increased thickness of granulation tissue prior to wound closure (47 versus 33 μm) though this was not statistically significant (p=0.25), increased thickness of epithelium prior to wound closure (220 versus 30 μm, p=0.04), increased thickness of keratin prior to wound closure (28 versus 10 μm, p<0.001), earlier spindle cell proliferation (Day 4 versus Day 8), less frequent separation of the epithelium from underling tissue (59% versus 100%, p=0.003), later neovascularization (Day 9-10 versus Day 8), and less frequent wound reopening (8% versus 48%, p<0.001).

Stages of wound healing of keratinocytes on muscle in the oral cavity:

inflammatory phase

granulation tissue

epithelial proliferation and migration

wound contraction

neovascularization

remodeling

TABLE 1

| | Control | | Treatment | |
|---|---|---|---|---|
| Stage | Timing | Comment | Timing | Comment |
| Inflammation | Day | | Day | |
| | 0-1 | | 0-1 | |
| Coverage by | Day | Granulation | Day | Granulation thickness |
| granulation | 2-5 | thickness less | 2-4 | greater |
| tissue | | 33 μm (SD 15) | | 47 μm (SD 6) |
| | | | | p = 0.25 |
| Epithelial | Day | Epithelial thickness | Day | Epithelial thickness |
| migration | 2-6 | less before closure | 2-4 | greater before closure |
| | | 30 μm (SD 0) | | 220 μm (SD 90) |
| | | Keratin thickness | | p = 0.04 |
| | | less before closure | | Keratin thickness |
| | | 10 μm (SD 0) | | greater before closure |
| | | | | 28 μm (SD 4) |
| | | | | p < 0.001 |
| Wound closure | Day | Happens later | Day | Happens earlier |
| | 6 | Epithelial thickness | 4/5 | P = 0.001 |
| | | 165 μm (SD 80) | | Epithelial thickness |
| | | Keratin thickness | | 153 μm (SD 33) |
| | | 23 μm (SD 5) | | Keratin thickness |
| | | | | 32 μm (SD 4) |
| Spindle cell | Day | Happens later | Day | Happens earlier |
| proliferation | 8 | | 4 | |
| Wound | Day | Epithelial separation | Day | Epithelial separation |
| contraction with | 6-9 | always (100%) | 4-8 | usually (58.9%) |
| epithelial | (up to 12) | occurs | | occurs |
| separation | | Height is greater | | P = 0.003 |
| | | 212 μm (SD 101) | | Height is less |
| | | Width is greater | | 130 μm (SD 92) |
| | | 515 μm (SD 277) | | p = 0.02 |
| | | Has a smaller | | Width is less |
| | | epithelial/keratin | | 358 μm (SD 243) |
| | | covering | | p = 0.10 |
| | | 132 μm (SD 52) | | Has a larger |
| | | | | epithelial/keratin |
| | | | | covering but not |
| | | | | significant |
| | | | | 185 μm (SD 73) |
| | | | | p = 0.09 |
| Neovascularization | Day | Happens during | Day | Happens after |
| | 7-9 | wound contraction | 9-10 | wound contraction + |
| | | and epithelial | | epithelial separation |
| | | separation | | |
| Wound reopening | Day | 48% of specimens | Day | 8% of specimens |
| | 8-14 | | 10-13 | p < 0.001 |

*Significant results are bolded

Discussion
1. Epithelial separation and wound contraction are potentially contributing factors to secondary PTH.
2. A HB-EGF-treated oral mouse wound showed greater epithelial and keratin thickness, less common epithelial separation, less common wound reopening, and earlier wound closure prior to neovascularization.
3. Neovascularization of the wound bed as the tissue heals may increase the risk of PTH. Epithelial separation and wound contraction happens after maximal neovascularization potential exposing new blood vessels to an unprotected surface. This did not occur in HB-EGF treated samples.
4. Thus, treatment with HB-EGF may prevent or decrease PTH.

### EXAMPLE 2

### Local Delivery of Heparin Binding-Epidermal Growth Factor After Tonsillectomy

When a tonsillectomy is performed, a raw wound is left in the oral cavity. Often at the wound bed is muscle and some lymphoid tissue. This wound granulates over the next few days and then epithelializes. It is hypothesized that neovascularization occurs in the wound prior to the epithelial layer becoming mature. This exposes the wound to secondary hemorrhage at this time (about day 5-10 post-tonsillectomy). This technology aims to delivery local HB-EGF to accelerate epithelialization of the wound so that this layer is mature prior to the neovascularization stage, which reduces the risk of secondary hemorrhage.

After surgery, a locally applied delivery vehicle containing HB-EGF with or without other bioactive substances (e.g., substances that promote epithelial adherence or decrease neoangiogenesis) can be directly applied to the wound. HB-EGF can be applied more than once. The vehicle may be bioabsorbable and release HB-EGF over time. A local anesthetic may also be administered in combination with the HB-EGF to reduce post-operative pain.

### EXAMPLE 3

### Delivery of Heparin Binding-Epidermal Growth Factor by Micro Needle Injection

After surgery and hemostasis has been achieved, a microneedle injection system containing HB-EGF can be placed onto the wound thereby causing the microneedles to deliver HB-EGF into the wound. The microneedles themselves may be bioabsorbable and release HB-EGF with or without other bioactive substances (e.g., substances that promote epithelial adherence or decrease neoangiogenesis) over time.

### EXAMPLE 4

### Local Delivery of Heparin Binding-Epidermal Growth Factor Adjacent to the Wound

After surgery, the delivery vehicle containing HB-EGF with or without other bioactive substances (e.g., substances that promote epithelial adherence or decrease neoangiogenesis) can be injected into or adjacent to the wound resulting from a tonsillectomy. The vehicle can be bioabsorbable and release HB-EGF over time.

### EXAMPLE 5

### Parenteral Delivery of Heparin Binding-Epidermal Growth Factor

After surgery, the delivery vehicle containing HB-EGF with or without other bioactive substances (e.g., substances that promote epithelial adherence or decrease neoangiogenesis) can be given to the patient via a non-trans oral route including parenteral or other systemic route so that HB-EGF localizes to and acts on the wound.

### EXAMPLE 6

### Oral Delivery of Heparin Binding-Epidermal Growth Factor

After surgery, the delivery vehicle containing HB-EGF with or without other bioactive substances (e.g., substances that promote epithelial adherence or decrease neoangiogenesis) can be given to the patient via a trans oral route as a wash, gargle, rinse, or topical paste so that the HB-EGF is applied to and acts on the wound.

### EXAMPLE 7

### Stability of HB-EGF in Human Saliva

Generally, growth factors and proteins are thought to be unstable in human saliva due to the presence of degrative enzymes, making them unsuitable for topical use in the oral cavity. To date, HB-EGF stability in saliva has not been investigated. Here, it was determined whether HB-EGF displayed any stability in saliva for potential use in therapy for oral wound healing.

Methods

SDS-PAGE Gels

Human saliva was filtered through a 0.45 μm syringe filter and then mixed with water and LDS sample buffer. HB-EGF was added. The solution was heated at 90° C. for 5 minutes and loaded on a 4-12% SDS-PAGE gel at physiologic pH.

Western Blot

Human saliva was mixed with water and buffer, and HB-EGF was added. The solution was run and antibodies to HB-EGF were used to monitor degradation of HB-EGF.

Results

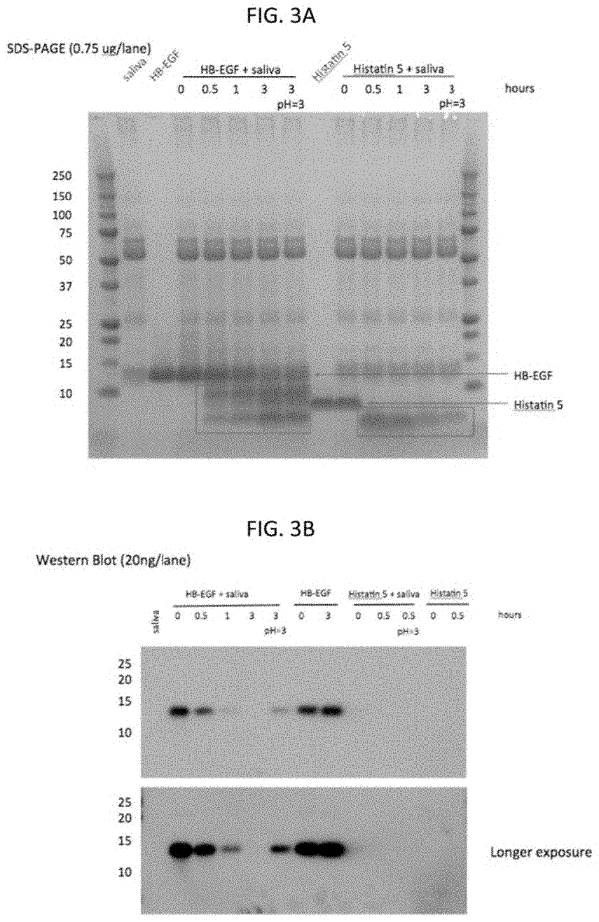

The SDS-PAGE gel shown in FIG. 3A demonstrates the presence of degradation products of HB-EGF after exposure to human saliva. Degradation products were visible by time=3 hours. Western blot results, shown in FIG. 3B, corroborate that virtually all HB-EGF was denatured by time=3 hours at physiologic pH. There is no endogenous HB-EGF in native saliva as demonstrated by Lane 1 in the Western blot. These results indicate that HB-EGF can last up to 3 hours in human saliva, during which time penetration could occur to deeper tissues, supporting its use in therapy for oral wound healing.

While the preferred embodiments of the invention have been illustrated and described, it will be appreciated that various changes can be made therein without departing from the spirit and scope of the invention

1. A method of treating a subject with an oral or oropharyngeal wound, the method
comprising administering a therapeutically effective amount of a composition comprising heparin binding epidermal growth factor (HB-EGF) to the subject.

2. The method of claim 1, wherein the wound is an oral wound.

3. The method of claim 1, wherein the subject is human.

4. The method of claim 1, wherein the HB-EGF is human HB-EGF.

5. The method of claim 1, wherein the HB-EGF promotes epithelialization of a wound produced by stimulating epithelial cell proliferation.

6. The method of claim 1, wherein treating the subject accelerates healing of the wound.

7. The method of claim 1, wherein treating the subject increases thickness of an epithelial layer at the wound.

8. The method of claim 1, wherein treating the subject increases rate of epithelialization at the wound.

9. The method of claim 1, wherein the composition is administered locally to the wound.

10. The method of claim 9, wherein the composition is administered by microneedle injection.

11. The method of claim 9, wherein the composition is administered by spraying the composition on the wound.

12. The method of claim 1, wherein the composition is administered orally, parenterally, or topically.

13. The method of claim 1, wherein the composition is administered adjacent to the site of the wound.

14. The method of claim 1, further comprising treating the subject with an antibiotic, an analgesic agent, an anti-inflammatory agent, an anesthetic, or another growth factor.

15. The method of claim 1, further comprising treating the subject with a substance that decreases neovascularization or a substance that improves adherence or decreases separation of the neoepithelium.

16. The method of claim 15, wherein the substance decreases contraction of muscle underlying the epithelium, whereby separation of the neoepithelium from the underlying musculature decreases.

17. The method of claim 1, wherein the composition further comprises a pharmaceutically acceptable carrier.

18. The method of claim 17, wherein the carrier is selected from the group consisting of an aqueous solution, a gel, a lotion, a balm, or a paste.

19. The method of claim 1, wherein multiple therapeutically effective doses of the HB-EGF are administered to the subject.

20. The method of claim 19, wherein multiple cycles of treatment are administered to the subject for a time period sufficient to effect at least a partial healing of the wound.

21-52. (canceled)


SEQUENCE LISTING (4 sequences)

SEQ ID NO: 1 - protein, 208 aa, Homo sapiens
  ref <308>: DATABASE ACCESSION NUMBER: NCBI/NP_001936
  ref <309>: DATABASE ENTRY DATE: 2015-03-15
  ref <313>: RELEVANT RESIDUES IN SEQ ID NO: (1)..(208)
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 2 - protein, 86 aa, Homo sapiens
DLQEADLDLLRVTLSSKPQALATPNKEEHGKRKKKGKGLGKKRDPCLRKYKDFCIHGECKYVKELRAPSCICHPGYHGERCHGLSL

SEQ ID NO: 3 - protein, 76 aa, Homo sapiens
RVTLSSKPQALATPNKEEHGKRKKKGKGLGKKRDPCLRKYKDFCIHGECKYVKELRAPSCICHPGYHGERCHGLSL

SEQ ID NO: 4 - protein, 75 aa, Homo sapiens
VTLSSKPQALATPNKEEHGKRKKKGKGLGKKRDPCLRKYKDFCIHGECKYVKELRAPSCICHPGYHGERCHGLSL